(19) United States
(12) Patent Application Publication
(10) Pub. No.: US 2015/0238614 A1
(43) Pub. Date: Aug. 27, 2015

(54) STABILISED AMORPHOUS FORM OF AGOMELATINE, A PROCESS FOR ITS PREPARATION AND PHARMACEUTICAL COMPOSITIONS CONTAINING IT

(71) Applicants: LES LABORATOIRES SERVIER, Suresnes (FR); SHANGHAI INSTITUTE OF PHARMACEUTICAL INDUSTRY, Shanghai (CN)

(72) Inventors: David Lafargue, Saint Jean Le Blanc (FR); Michael Lynch, Saint Jean De La Ruelle (FR); Cécile Poirier, Orleans (FR); Philippe Letellier, Orleans (FR); Jean-Manuel Pean, Orleans (FR); Ying Luo, NanChang City (CN); Hanbin Shan, Gaoan (CN); Yuhui Shen, Shanghai (CN)

(21) Appl. No.: 14/426,853

(22) PCT Filed: Sep. 11, 2013
(86) PCT No.: PCT/EP2013/068792

§ 371 Date: Mar. 10, 2015

(30) Foreign Application Priority Data
Sep. 11, 2012 (CN) PCT/CN2012/081250
Sep. 26, 2012 (FR) 12/59064

(51) Int. Cl. A61K 47/38; B29C 47/00; A61K 47/34; A61K 31/165; A61K 47/32

(52) U.S. Cl. CPC A61K 47/38; A61K 31/165; A61K 47/32; A61K 47/34; B29C 47/0066; B29K 2079/00

(57) ABSTRACT

Stabilised amorphous form of the compound of formula (I):

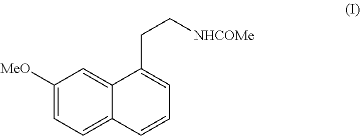

Medicaments

The present invention relates to the stabilised amorphous form of agomelatine, or N-[2-(7-methoxy-1-naphthyl)ethyl]acetamide of formula (I):

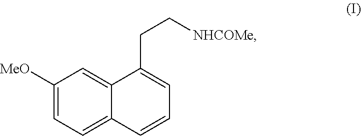

to a process for its preparation and also to pharmaceutical compositions containing it.

Agomelatine, or N-[2-(7-methoxy-1-naphthyl)ethyl]acetamide, has valuable pharmacological properties.

In fact, it has the double characteristic of being, on the one hand, an agonist of receptors of the melatoninergic system and, on the other hand, an antagonist of the 5-HT_{2C} receptor. These properties provide it with activity in the central nervous system and, more especially, in the treatment of major depression, seasonal affective disorder, sleep disorders, cardiovascular pathologies, pathologies of the digestive system, insomnia and fatigue due to jet-lag, appetite disorders and obesity.

Agomelatine, its crystalline forms, its complexes, its co-crystals, its addition salts with a pharmaceutically acceptable acid or base, its preparation and its use in therapeutics have been described in the patent applications EP0447285, WO2005/077887, WO2007/015003, WO2007/015002, WO2007/015004, WO2010/097052, WO2010/102554, CN101955440, WO2011/050742, CN102050756, WO2011/006387, WO2011/075943, CN101774937, WO2011/006387, CN101870662 and CN102030673.

Agomelatine is moreover an active ingredient which has the disadvantage of having a very low bioavailability and, as a consequence, a large inter-individual variability. This low bioavailability is in part related to the solubility of agomelatine in water, which is less than 0.15 mg/ml at 25° C.

In view of the pharmaceutical value of this compound and its poor bioavailability, the amorphous form seems to be a possible strategy, as amorphous forms of solid compounds are known to have better dissolution properties than their corresponding crystalline forms.

The Applicant has now discovered that agomelatine has an amorphous state whose glass transition temperature has been measured at −6° C. by scanning calorimetric analysis, or DSC (“Differential Scanning calorimetry”). Above that temperature, the active ingredient undergoes a transition towards a crystalline form. Preserving agomelatine in the amorphous state at ambient temperature is therefore not possible.

Accordingly, the technical problem posed is to make available the amorphous form of agomelatine in a formulation which is compatible with its use in the pharmaceutical industry, especially in terms of stability and storage under customary conditions.

A solution known to the person skilled in the art consists of forming a dispersion of the active ingredient in a solid matrix which surrounds the molecules and prevents them from forming a crystal lattice. The more dilute the active ingredient within the matrix, the greater the amorphous formation. A consequence is that the size of tablets containing the amorphous active ingredient increases substantially with the matrix, which constitutes a major drawback for the patient who has to swallow them. Accordingly, another challenge lies in minimising the amount of matrix used, to the benefit of the active ingredient, whilst still preventing the crystal lattice from forming.

Tests have been carried out with agomelatine in cyclodextrins or trehalose, which are customarily used to form complexes of the “host-guest” type, but it has never been possible to obtain, in a manner that is stable over time, the type of complexes sought.

The Applicant has now discovered that it is possible to obtain a stable formulation of agomelatine in amorphous form in reproducible and industrially feasible manner. This new stabilised form allows to envisage its use in the pharmaceutical industry. It moreover allows high active ingredient contents, resulting in a final size of formulation which is entirely compatible with its being taken by the patient.

The present invention thus relates to the stabilised amorphous form of agomelatine. By “stabilised” it is understood that the amorphous form of agomelatine is preserved when it is subjected to denaturing storage conditions of temperature and humidity for at least one week. The denaturing conditions of temperature and humidity according to the invention will be on average, for example 40° C./75% RH (relative humidity), 30° C./65% RH, 50° C., 70° C.

More specifically, the present invention consists of a solid dispersion of agomelatine within an organic polymer, it being understood that the agomelatine represents at least 30% by weight of the dispersion. It is possible, depending on the organic polymer chosen, to increase this percentage by weight of agomelatine. The solid dispersions wherein the percentage by weight of agomelatine is from 30 to 50%, and more preferably from 30 to 40% are preferred. Surprisingly, even at those high active ingredient contents, the amorphous form of agomelatine is preserved—in a manner that is stable over time. Moreover, besides the improvement in dissolution rate which could be expected customarily with amorphisation of the active ingredient, the solubility of the agomelatine obtained is also greatly increased well beyond the solubility measured for crystalline agomelatine, with its being at least tripled and in some cases multiplied by a factor of 8. This result is entirely surprising because although numerous publications in the field describe an improvement in the biopharmaceutical performance of active ingredients (solubility, dissolution rate) by designing stable solid dispersions this is for loadings of less than 20% of active ingredient (Lin et al., International Journal of Pharmaceutics, 1996, 127, 261-272).

Surprisingly, the increase in solubility of the active ingredient is maintained for at least 4 hours.

The polymer used in the present invention relates more especially to methacrylic acid compounds or vinyl or cellulosic polymers.

More especially, the polymers used according to the invention relate to polymethacrylates or copolymers of methacrylic acid which correspond to a completely polymerised copolymer of methacrylic acid and acrylic or methacrylic ester. These polymethacrylates are customarily referred to as Eudragit® and may be in the form of a powder or granules. Among the different Eudragit® products marketed, those preferably used in the context of the invention are Eudragit® L products, more especially Eudragit® L100 and L100-55, or Eudragit® EPO. These polymers are particularly adapted to all ranges of agomelatine loading (% by weight of agomelatine).

The vinyl polymers according to the invention relate more especially to polyvinyl esters such as, for example, polyvinyl acetate phthalate; homo- or co-polymers polyvinylpyrrolidone based such as Povidones (Povidone® K30, Plasdone® S630), Kollidon VA64 or also Soluplus®. Povidones will be used for loading inferior to 50% by weight of agomelatine.

Among the cellulosic compounds used according to the invention there may be mentioned cellulosic ethers or esters such as HPMCs, and more especially HPMC acetyl succinate.

The present invention relates also to a process for obtaining a stable formulation having a high content of the amorphous form of agomelatine within a polymer. According to the process of the invention, the compound of formula (I) obtained by any process and present in any crystalline form, complexes, co-crystals, or addition salts with a pharmaceutically acceptable acid or base, is mixed with the selected polymer in one or more solvents, allowing complete dissolution of the constituents to be obtained, and then the solvent is evaporated off in its entirety under reduced pressure.

The solvents used according to the invention are those capable of dissolving agomelatine and the selected polymer; preference will be given to polar protic or aprotic solvents such as acetone, alcohols and more especially methanol and ethanol, water, dichloromethane, ethyl acetate or mixtures of those solvents. Dissolution is carried out with stirring at ambient temperature or by heating the mixture until dissolution of the constituents is complete. Evaporating off the solvents is carried out under reduced pressure at ambient temperature or by heating until evaporation of the solvents is complete.

An advantageous embodiment of the process for preparation of a stable formulation having a high content of the amorphous form of agomelatine within a polymer consists of mixing and pre-blending the compound of formula (I) obtained by any process and present in any crystalline form, complexes, co-crystals, or addition salts with a pharmaceutically acceptable acid or base, with the selected polymer and then this mixture is introduced into an extruder whose screw pitch and temperature are chosen as a function of the viscosity of the mixture, to obtain an extrudate which is then cut up into the desired size and then, optionally, ground.

Preferably, rotation of the screw will be carried out between 50 and 200 rpm, more especially between 75 and 150 rpm.

The extrusion temperature selected will be a function of the viscosity of the resulting mixture of constituents and will be between 90° C. and 200° C. inclusive.

In the processes for obtaining the stabilised amorphous form of agomelatine according to the invention, the compound of formula (I) obtained by any process and present in any crystalline form, complexes, co-crystals, or addition salts with a pharmaceutically acceptable acid or base, may be used.

In the processes according to the invention, the agomelatine loading is greater than or equal to 30% by weight and more especially varies from 30 to 50% by weight, preferably from 30% to 40%.

The stabilised amorphous form thereby obtained has value in the treatment of disorders of the melatoninergic system and has shown substantial activity on the central nervous system and microcirculation, making it possible to establish its usefulness in the treatment of stress, sleep disorders, anxiety, major depression, seasonal affective disorder, bipolar disorder, generalised anxiety disorder, cardiovascular pathologies, pathologies of the digestive system, insomnia and fatigue due to jet-lag, schizophrenia, panic attacks, melancholia, appetite disorders, obesity, insomnia, pain, psychotic disorders, epilepsy, diabetes, Parkinson's disease, senile dementia, various disorders associated with normal or pathological ageing, migraine, memory loss, Alzheimer's disease, and also in cerebral circulation disorders. In another field of activity, it appears that, in treatment, the amorphous form of agomelatine can be used in sexual dysfunctions, that it has ovulation-inhibiting and immunomodulating properties and that it may potentially be used in the treatment of cancers.

The stabilised amorphous form of agomelatine will preferably be used in treating major depression, seasonal affective disorder, bipolar disorder, generalised anxiety disorder, sleep disorders, cardiovascular pathologies, pathologies of the digestive system, insomnia and fatigue due to jet-lag, appetite disorders and obesity.

Obtaining the stabilised amorphous form of agomelatine has the advantage of making possible the preparation of pharmaceutical formulations of consistent and reproducible composition, which have excellent stability over time.

According to the invention it is therefore possible to obtain solid pharmaceutical compositions having high contents of the amorphous form of agomelatine, which are administrable especially by the oral, buccal, sublingual, ocular, rectal, vaginal or parenteral route.

These pharmaceutical compositions can be made of the solid dispersion of agomelatine within the polymer without any other processing operation besides packaging. If desired, said pharmaceutical compositions may, however, undergo processing by grinding or by granulation for filling into capsules or for compression or may undergo coating.

The pharmaceutical compositions of the invention may also optionally comprise pharmacologically acceptable excipients selected, for example, from the group of binders, disintegrating agents, disintegrants, lubricants, diluents, antioxidants, aromatic agents, colourants, preservatives, sweeteners and anti-adherents.

Among the pharmaceutical compositions according to the invention there may be more especially mentioned tablets or dragées, granules, sublingual tablets, capsules, lozenges, suppositories, creams, ointments, dermal gels, injectable preparations, drinkable suspensions and chewing gums.

Preferably, the pharmaceutical compositions according to the invention contain at least 25% by weight of agomelatine with respect to the total weight of the formulation.

The useful dosage can be varied according to the nature and severity of the disorder, the administration route and the age and weight of the patient. The dosage varies from 0.1 mg to 1 g per day of agomelatine in one or more administrations.

### DESCRIPTION OF THE FIGURES

FIG. 1: X-ray diffraction diagrams of Example 10 recorded under various stability conditions (1 week at 40° C. 75% RH, 1 week at 40° C./75% RH then 1 week at 50° C., 1 week at 70° C.)

FIG. 2: X-ray diffraction diagrams of Example 11 recorded under various stability conditions (1 week at 40° C. 75% RH, 1 week at 40° C./75% RH then 1 week at 50° C., 1 week at 70° C.)

FIG. 3: X-ray diffraction diagrams of Example 12 recorded under various stability conditions (1 week at 40° C. 75% RH, 1 week at 40° C./75% RH then 1 week at 50° C., 1 week at 70° C.)

FIG. 4: X-ray diffraction diagrams of Example 19 recorded under various stability conditions (1 week at 40° C. 75% RH, 1 week at 40° C./75% RH then 1 week at 50° C., 1 week at 70° C.)

FIG. 5: X-ray diffraction diagrams of Example 28 recorded under various stability conditions (1 week at 40° C. 75% RH, 1 week at 40° C./75% RH then 1 week at 50° C., 1 week at 70° C.)

FIG. 6: X-ray diffraction diagrams recorded at the end of 3 months under various stability conditions (25° C./60% RH in an open or closed flask, 30° C./65% RH in an open or closed flask, 50° C. in an open flask) of Example 39

FIG. 7: X-ray diffraction diagrams recorded at the end of 3 months under various stability conditions (25° C./60% RH in an open or closed flask, 30° C./65% RH in an open or closed flask, 50° C. in an open flask) of Example 40

The Examples hereinbelow illustrate the invention but do not limit it in any way.

### A. General Procedure for Obtaining the Stabilised Amorphous Form of Agomelatine by Dissolution-Evaporation

Agomelatine and the selected polymer are placed in a 10-ml vial with 3 ml of solvent(s). The mixture is stirred at 40° C. for 30 minutes until dissolution is complete and a homogeneous solution has been obtained. The solvent(s) is/are evaporated off under reduced pressure at 40° C. for 30 minutes. The residue obtained is finally dried in vacuo at ambient temperature (20° C.) overnight (12 hours) to yield the stabilised amorphous form of agomelatine. For each test, the stability of the amorphous form was tested under denaturing conditions at 40° C. and 75% RH, at 40° C. and 75% RH followed by 50° C., and also at 70° C.

The various Examples produced and results obtained are set out in the Table below:

| | | | | Stability under denaturing conditions | | |
|---|---|---|---|---|---|---|
| | | | | | 1 week at | |
| | | | | | 40° C., 75% RH, | |
| | Agomelatine | | | 1 week at | then 1 week at | 1 week at |
| Example | % by weight | Polymer | Solvents | 40° C., 75% RH | 50° C. | 70° C. |
| 1 | 30 | Eudragit | Acetone | amorphous | amorphous | amorphous |
| 2 | | L100-55 | EtOH | | | |
| 3 | | | Acetone/EtOH | | | |
| | | | 70/30 | | | |
| 4 | 35 | | Acetone | amorphous | amorphous | amorphous |
| 5 | | | EtOH | | | |
| 6 | | | Acetone/EtOH | | | |
| | | | 70/30 | | | |
| 7 | 40 | | Acetone | amorphous | amorphous | amorphous |
| 8 | | | EtOH | | | |
| 9 | | | Acetone/EtOH | | | |
| | | | 70/30 | | | |
| 10 | 50 | | Acetone | amorphous | amorphous | amorphous |
| 11 | | | EtOH | | | |
| 12 | | | Acetone/EtOH | | | |
| | | | 70/30 | | | |
| 13 | 30 | Kollidon | Acetone | amorphous | amorphous | amorphous |
| 14 | | VA 64 | EtOH | | | |
| 15 | | | Acetone/EtOH | | | |
| | | | 70/30 | | | |
| 16 | 35 | | Acetone | amorphous | amorphous | amorphous |
| 17 | | | EtOH | | | |
| 18 | | | Acetone/EtOH | | | |
| | | | 70/30 | | | |
| 19 | 40 | | Acetone | amorphous | amorphous | amorphous |
| 20 | | | EtOH | | | |
| 21 | | | Acetone/EtOH | | | |
| | | | 70/30 | | | |
| 22 | 30 | Soluplus | Acetone | amorphous | amorphous | amorphous |
| 23 | | | EtOH | | | |
| 24 | | | Acetone/EtOH | | | |
| | | | 70/30 | | | |
| 25 | 35 | | Acetone | amorphous | amorphous | amorphous |
| 26 | | | EtOH | | | |
| 27 | | | Acetone/EtOH | | | |
| | | | 70/30 | | | |
| 28 | 40 | | Acetone | amorphous | amorphous | amorphous |
| 29 | | | EtOH | | | |
| 30 | | | Acetone/EtOH | | | |
| | | | 70/30 | | | |

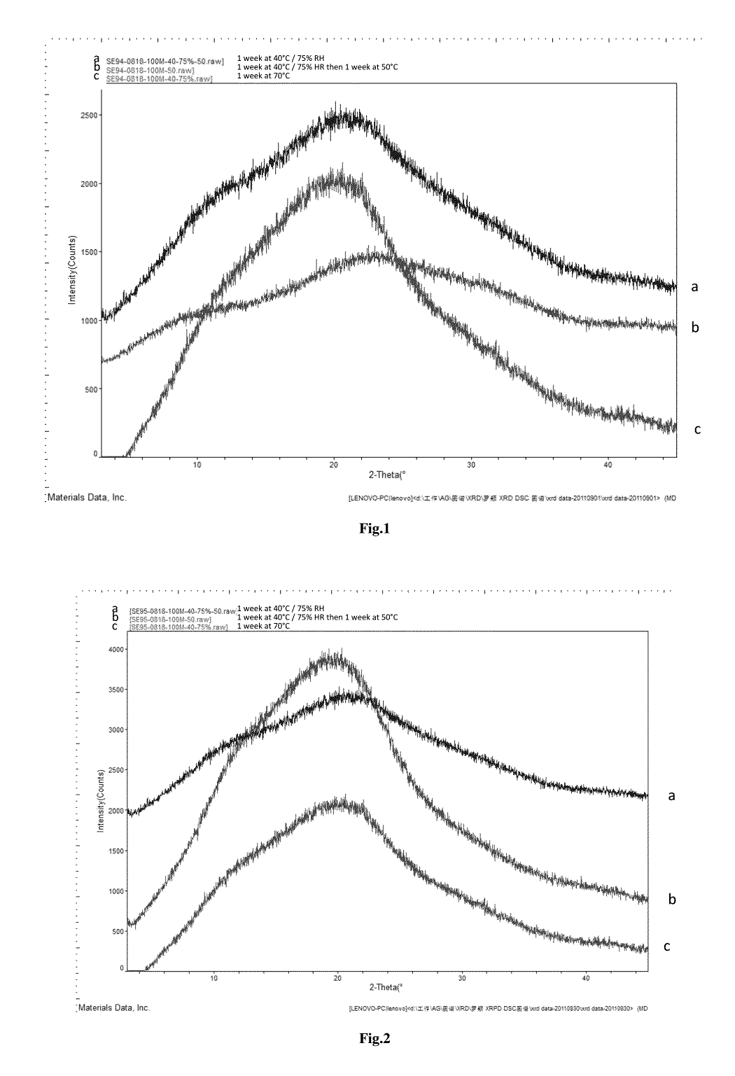

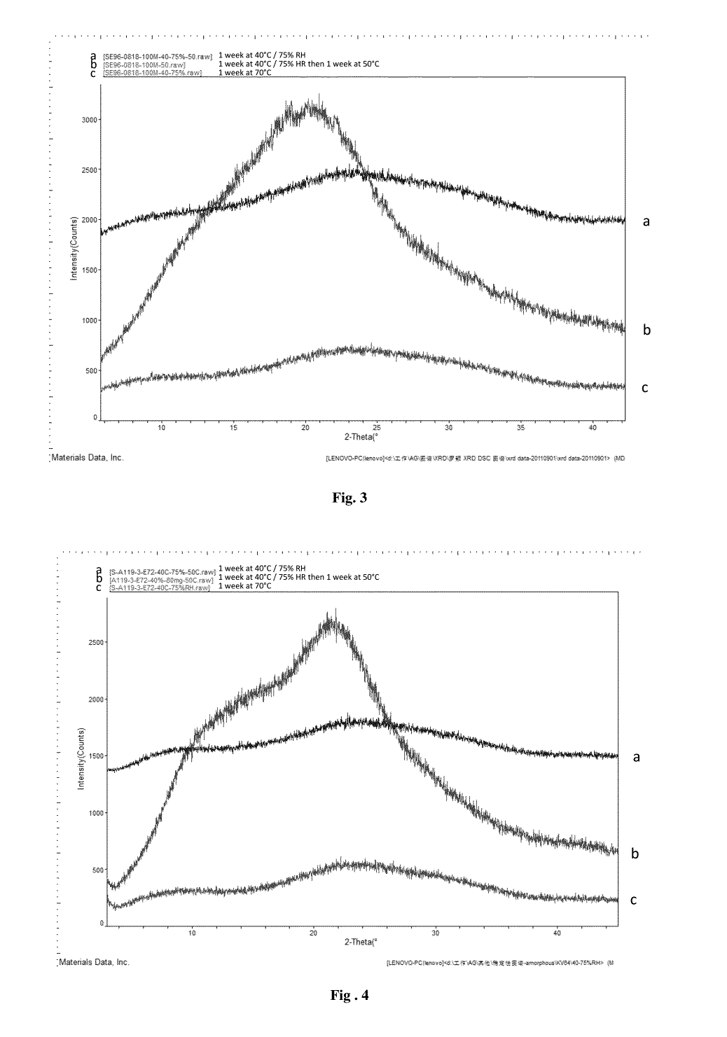

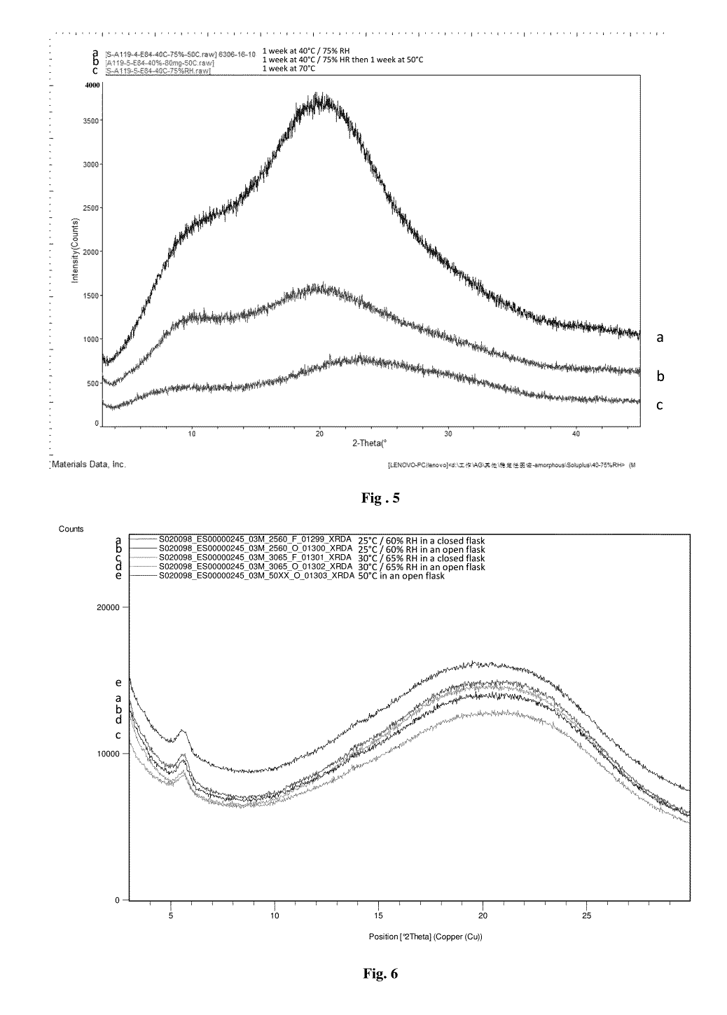

By way of example, the X-ray diffraction diagrams of examples 10, 11, 12, 19 and 28 recorded under various stability conditions (1 week at 40° C. 75% RH, 1 week at 40° C./75% RH then 1 week at 50° C., 1 week at 70° C.) are set out in FIG. 1, FIG. 2, FIG. 3, FIG. 4 and FIG. 5 respectively.

### B. General Process for Obtaining the Stabilised Amorphous Form of Agomelatine by Extrusion

Agomelatine and the polymer are pre-blended in a Turbula-type mixer for 10 minutes. The mixture obtained is placed manually in a conical rotor extruder (diameter 5/14 mm) of the HAAKE Minilab II Microcompounder type (ThermoFisher). The extrusion speed is 100 rpm.

The stability of the amorphous form obtained was assessed under various temperature and relative humidity conditions: 25° C./60% RH in an open or closed flask, 30° C./65% RH in an open or closed flask, 50° C. in an open flask.

All the Examples given in the Table below have stabilities of more than at least 6 weeks.

| | Agomelatine | | Extrusion |
|---|---|---|---|
| | % by weight | Polymer | temperature |
| 31 | 30 | Eudragit L100 | 190° C. |
| 32 | 40 | | |
| 33 | 50 | | |
| 34 | 30 | Eudragit L100-55 | 150° C. |
| 35 | 40 | | |
| 36 | 50 | | |
| 37 | 30 | HPMC acetyl succinate | 135° C. |
| 38 | 30 | Plasdone S630 | 110° C. |
| 39 | 30 | Polyvinyl acetate phthalate | 150° C. |
| 40 | 40 | | |
| 41 | 30 | Povidone K30 | 140° C. |

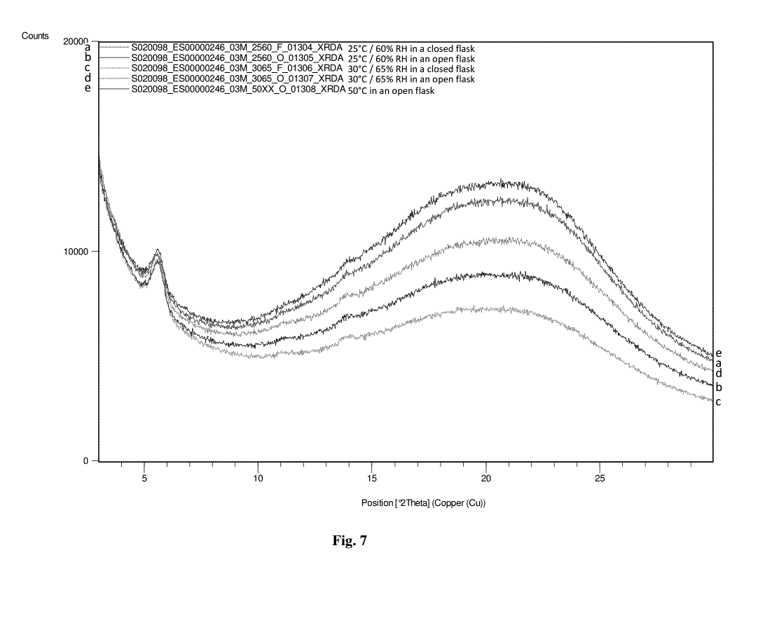

By way of example, the X-ray diffraction diagrams recorded at the end of 3 months under various stability conditions (25° C./60% RH in an open or closed flask, 30° C./65% RH in an open or closed flask, 50° C. in an open flask) for Examples 39 and 40 are set out in FIG. 6 and FIG. 7 respectively.

### C. Solubility

A solubility study of the formulations obtained was carried out using a Crystal16® type apparatus in a pH 6.8 buffer solution at 25° C. over a period of 4 hours with a stirring speed of 700 rpm. Various concentrations were tested and the presence of insoluble particles was monitored by nephelometric detection. As reference, the solubility of agomelatine under those conditions is 0.14 mg/ml.

The results obtained are set out in the Table below, which sets out i) the maximum solubility observed and the time at which this maximum solubility is observed, ii) the solubility observed at 4 hours.

| | Max. solubility (mg/ml) | Solubility |
|---|---|---|
| Example | observed at t minutes | (mg/ml) at 4 hours |
| 14 | >0.9 at 5 mins. | >0.59 |
| 20 | >0.9 at 6 mins. | >0.42 |
| 31 | >1.5 at 5 mins. | >1.20 |
| 32 | >1.2 at 45 mins. | >0.46 |
| 33 | >1.1 at 10 mins. | >0.52 |
| 34 | >0.67 at 5 mins. | >0.24 |
| 35 | >0.9 at 5 mins. | >0.40 |
| 36 | >0.76 at 10 mins. | >0.33 |
| 37 | >0.79 at 5 mins. | >0.32 |
| 38 | >0.34 at 5 mins. | >0.34 |
| 39 | >0.64 at 5 mins. | >0.43 |
| 40 | >0.53 at 5 mins. | >0.29 |
| 41 | >0.73 at 5 mins. | >0.37 |

The results obtained show a substantial increase in the maximum solubility. The important point is that this solubility increase continues over time: at 4 hours, the results show that it is at least multiplied by a factor ranging from 1.7 to 8.5, which gives time for the active ingredient to be absorbed before it is reprecipitated.

D. Pharmaceutical compositions

### Example 42

Formula for the preparation of 1000 capsules each containing 25 mg of agomelatine:

| | |
|---|---|
| Agomelatine | 25 g |
| Eudragit L100-55 | 25 g |

The extrudate is prepared in accordance with Example 36 and is then cut into a mini-matrix and introduced into a size 1 capsule.

### Example 43

Formula for the preparation of 1000 capsules each containing 25 mg of agomelatine:

| | |
|---|---|
| Agomelatine | 25 g |
| Plasdone S630 | 58 g |

The extrudate is prepared in accordance with Example 38 and is then cut into a mini-matrix and introduced into a size 1 capsule.

### Example 44

Formula for the preparation of 1000 tablets each containing 25 mg of agomélatine:

| | | |
|---|---|---|
| Extrudat Exemple 36 | 50 | g |
| Maize starch | 10 | g |
| Lactose | 20 | g |
| Magnésium stearate | 0.5 | g |
| Silica | 0.25 | g |
| Hydroxypropylcellulose | 2.25 | g |

1-26. (canceled)

27. A stabilised amorphous form of agomelatine of formula (I):

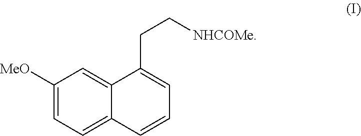

28. The stabilised amorphous form of agomelatine according to claim 27, wherein the agomelatine is dispersed within a matrix.

29. The stabilised amorphous form of agomelatine according to claim 28, wherein the matrix used is an organic polymer.

30. The stabilised amorphous form of agomelatine according to claim 28, wherein the matrix used is a methacrylic acid compound or a vinyl or cellulosic polymer.

31. The stabilised amorphous form of agomelatine according to claim 30, wherein the matrix used is Eudragit L100.

32. The stabilised amorphous form of agomelatine according to claim 30, wherein the matrix used is Eudragit L100-55.

33. The stabilised amorphous form of agomelatine according to claim 28, wherein the matrix used is a vinyl polymer.

34. The stabilised amorphous form of agomelatine according to claim wherein the matrix used is Kollidon VA64 or Soluplus.

35. The stabilised amorphous form of agomelatine according to claim 33, wherein the matrix used is Polyvinyl Acetate Phthalate.

36. The stabilised amorphous form of agomelatine according to claim 33, wherein the matrix used is Plasdone 8630.

37. The stabilised amorphous form of agomelatine according to claim 28, wherein the matrix used is a cellulosic polymer.

38. The stabilised amorphous form of agomelatine according to claim 37, wherein the matrix used is HPMC acetyl succinate.

39. The stabilised amorphous form of agomelatine according to claim 28, wherein the percentage of agomelatine by weight that is used is greater than or equal to 30%.

40. The stabilised amorphous form of agomelatine according to claim 28, wherein the percentage of agomelatine by weight that is used is from 30% to 50%.

41. The stabilised amorphous form of agomelatine according to claim 28, wherein the percentage of agomelatine by weight that is used is from 30% to 40%.

42. A process for obtaining the stabilised amorphous form of agomelatine according to claim 27, wherein the compound of formula (I) obtained by any process and present in any crystalline form, complexes, co-crystals, or addition salts with a pharmaceutically acceptable acid or base, is mixed with a polymer in one or more solvents, allowing complete dissolution of the constituents to be obtained, and then the solvent is evaporated off in its entirety under reduced pressure.

43. A process for obtaining the stabilised amorphous form of agomelatine according to claim 27, wherein the compound of formula (l) obtained by any process and present in any crystalline form, complexes, co-crystals, or addition salts with a pharmaceutically acceptable acid or base, is mixed and pre-blended with a polymer and then is introduced into an extruder whose screw pitch and temperature are chosen as a function of the viscosity of the mixture, to obtain an extrudate which is then cut up into the desired size and then, optionally, ground.

44. The process according to claim 42, wherein the percentage of agomelatine by weight is greater than or equal to 30%.

45. The process according to claim 42, wherein the percentage of agomelatine by weight is from 10% to 50%.

46. The process according to claim 42, wherein the percentage of agomelatine weight is from 30% to 40%.

47. The process according to claim 43, wherein the percentage of agomelatine by weight is greater than or equal to 30%.

48. The process according to claim 43, wherein the percentage of agomelatine by weight is from 30% to 50%.

49. The process according to claim 43, wherein the percentage of agomelatine by weight is from 30% to 40%.

50. A pharmaceutical composition comprising as active ingredient the stabilised amorphous form of agomelatine according to claim 27, alone or in combination with one or more inert, non-toxic and pharmaceutically acceptable carriers.

51. The pharmaceutical composition according to claim 50, wherein the percentage of agomelatine by weight with respect to the total weight of the formulation is greater than or equal to 25%.

52. A method of treating disorders of the melatoninergic system in a subject in need thereof, comprising administration of the stabilised morphous form of agomelatine according to claim 27, alone or in combination with one or more inert, non-toxic and pharmaceutically acceptable carriers.

53. A method of treating a condition selected from sleep disorders, stress, anxiety, seasonal affective disorder or major depression, cardiovascular pathologies, pathologies of the digestive system, insomnia and fatigue due to jet-lag, schizophrenia, panic attacks, melancholia, appetite disorders, obesity, insomnia, pain, psychotic disorders, epilepsy, diabetes, Parkinson's disease, senile dementia, various disorders associated with normal or pathological ageing, migraine, memory loss, Alzheimer's disease, cerebral circulation disorders, in sexual dysfunctions, conditions requiring, an ovulation-inhibitor, conditions requiring an immunomodulator, and cancer in a subject in need thereof, comprising administration of the stabilised amorphous form of agomelatine according to claim 27, alone or in combination with one or more inert, non-toxic and pharmaceutically acceptable carriers.
